(19) United States
(12) Patent Application Publication
(10) Pub. No.: US 2018/0072973 A1
(43) Pub. Date: Mar. 15, 2018

(54) AUTOLOGOUS CELL MULTIDIMENSIONAL MOLDING APPARATUS

(71) Applicant: 3D GLOBAL BIOTECH INC., New Taipei City (TW)

(72) Inventors: KENG-LIANG OU, New Tapei City (TW); CHIEN-HUI HUNG, New Tapei City (TW); YU-HAO CHAN, New Taipei City (TW)

(21) Appl. No.: 15/702,551

(22) Filed: Sep. 12, 2017

(30) Foreign Application Priority Data
Sep. 13, 2016 (TW) 105129810

(51) Int. Cl. C12M 3/00; C12M 1/00; C12M 1/34; C12M 1/32; B33Y 30/00; B33Y 50/02; B29C 64/106; B33Y 80/00

(52) U.S. Cl. CPC C12M 21/08; C12M 29/12; C12M 41/40; C12M 43/00; B29L 2031/753; B33Y 30/00; B33Y 50/02; B29C 64/106; B33Y 80/00; C12M 33/06

(57) ABSTRACT

The present invention discloses an autologous cell multidimensional molding apparatus, which can customize an artificial implant by using an autologous cell. The autologous cell multidimensional molding apparatus comprises a barotropic room, an autologous cell culturing module, a supplying module, a multi-axis molding module, and an implant saving module. The autologous cell culturing module cultivates the autologous cells and sends the autologous cells to the supplying module to mix with polymer materials to be combined autologous cells. The multi-axis molding module prints the combined autologous cells to be artificial implants for carrying and saving the combined autologous cells by the implant saving module. Wherein, the barotropic room is filled with a gas having pressure greater than an ambient pressure. The modules listed above are all inside the barotropic room and connected by closed pipes. Thus, compared to the prior art, the present invention can customize the artificial implants and effectively enhance the yield of the preparation of the artificial implants.

### CROSS-REFERENCE TO RELATED APPLICATIONS

This application claims priority to Taiwan Patent Application No. 105129810, filed Sep. 13, 2016, reference of which is hereby incorporated in its entirety.

### BACKGROUND OF THE INVENTION

### 1. Field of the Invention

The present invention relates to a molding apparatus, and more particularly, to an autologous cell multidimensional molding apparatus.

### 2. Description of the Prior Art

With the advancement of medical technology, the use of artificial implants such as artificial dressings and artificial organs is becoming more frequent. Artificial dressings are commonly used in a wide range of burns or postoperative patients to replace the damaged skin, protect the wound from contaminants or bacterial infection, absorb their body fluids to keep the body moisture from escaping and promote the growth of connective tissue. Artificial organs such as artificial electronic ears or artificial blood vessels can be used to simulate and replace the function of the original organ, or semi-support with the original organ to maintain the body function.

However, artificial implants may also cause adverse reactions to the human body resulting in exclusion of the human body due to heterogeneous substances and additional immune mutex problems. Therefore, autologous cells are used as the substrate of artificial implants later.

The multidimensional molding method of autologous tissue is carried out by filling a raw material or a cell material in a bucket, putting into a raw material section of multidimensional molding apparatus and extracting from the raw material holder to perform the formation of the cells and tissues of the artificial implants.

During the process, the raw materials are easy to be contaminated because of human movement and the replacement of vessels, which results in not high implant molding yield. Besides, as multidimensional molding apparatus is in an open space, such conventional process makes the manufacture of the artificial implants susceptible to be contaminated due to persons or the environment, which further results in not high implant molding products. Therefore, how to design a cell tissue multidimensional molding apparatus that the raw materials for molding and the forming process are not contaminated by the outside world is an urgent problem to be solved now.

The above-mentioned conventional technology still has a lot of missing, and it is not a good design; therefore, it needs to be improved. In view of the above, the present invention provides an autologous cell multidimensional molding apparatus to facilitate efficient production.

### SUMMARY OF THE INVENTION

An object of the present invention is to provide an autologous cell multidimensional molding apparatus. According to an embodiment of the present invention, the autologous cell multidimensional molding apparatus is used for customizing an artificial implant by using an autologous cell, which comprises a barotropic room, an autologous cell culturing module, a supplying module, a multi-axis molding module and an implant saving module. The barotropic room is used for inputting a gas having a pressure greater than an ambient pressure. The autologous cell culturing module is configured in the barotropic room and comprises a culture vessel and a feed pipe, wherein the culture vessel is used for culturing the autologous cell into a cultured autologous cell, and the feed pipe is connected to the culture vessel for receiving the cultured autologous cell. The supplying module is configured in the barotropic room and comprises a peristaltic feeder and a discharge pipe, wherein the peristaltic feeder is connected to the feed pipe for receiving the cultured autologous cell and mixing the cultured autologous cell with a polymer material to be a combined autologous cell, and the discharge pipe is connected to the peristaltic feeder for receiving the combined autologous cell. The multi-axis molding module is configured in the barotropic room, and the multi-axis molding module is connected to the discharge pipe for receiving the combined autologous cell and customizing to print the combined autologous cell to the artificial implant. The implant saving module is configured in the barotropic room and is set under the multi-axis molding module for carrying and saving the artificial implant. Wherein, the feed pipe and the discharge pipe are enclosed pipelines respectively.

Furthermore, the connection between the feed pipe and the culture vessel, the connection between the peristaltic feeder and the feed pipe, the connection between the discharge pipe and the peristaltic feeder and the connection between the multi-axis molding module and the discharge pipe are enclosed pipelines respectively.

The multi-axis molding module comprises a printing material injection head, a pattern receiving element and a six-axis moving arm. The printing material injection head is connected to the discharge pipe for inputting the combined autologous cell and printing the artificial implant. The pattern receiving element is used for receiving a customized pattern, and the six-axis moving arm is electrically connected to the pattern receiving element for driving a customized printing itinerary of the printing material injection head.

Wherein, the six-axis moving arm moves in a three-dimensional space with an angular movement.

As compared to the prior art, the autologous cell multidimensional molding apparatus of the present invention uses the barotropic room to produce a production environment of pollution-free, and uses the closed connection to isolate the whole production process from the autologous cell material to the artificial implant product from the outside world, and uses the six-axis molding module to make a customized autologous cell multidimensional print to get the high yield of the customized artificial implant. Also, the implant holding device can be used to make an artificial implant of flexible time and place.

The advantages and spirits of the invention may be understood by the following recitations together with the appended drawings.

### BRIEF DESCRIPTION OF THE APPENDED DRAWINGS

Some of the embodiments will be described in detail, with reference to the following figures, wherein like designations denote like members, wherein:

FIG. 1 shows a schematic diagram of the autologous cell multidimensional molding apparatus in an embodiment of the present invention.

FIG. 2 shows a function block diagram of the autologous cell multidimensional molding apparatus in an embodiment of the present invention.

FIG. 3 shows a schematic diagram of the culture vessel of the autologous cell multidimensional molding apparatus in an embodiment of the present invention.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

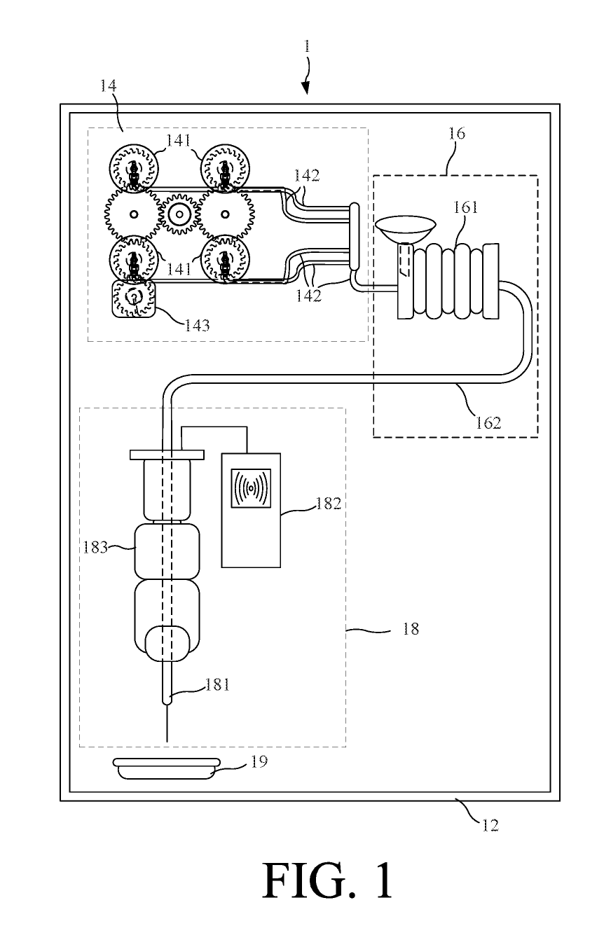

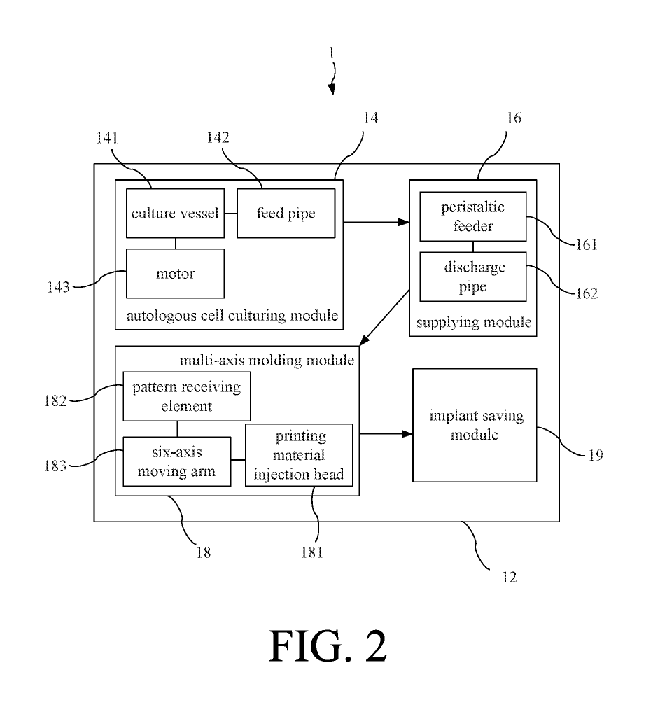

Please refer to FIG. 1 and FIG. 2. FIG. 1 shows a schematic diagram of the autologous cell multidimensional molding apparatus 1 in an embodiment of the present invention. FIG. 2 shows a function block diagram of the autologous cell multidimensional molding apparatus 1 in an embodiment of the present invention. In an embodiment of the present invention, the autologous cell multidimensional molding apparatus 1 comprises a barotropic room 12, an autologous cell culturing module 14, a supplying module 16, a multi-axis molding module 18 and an implant saving module 19. The barotropic room 12 is used for inputting a gas having a pressure greater than an ambient pressure. The autologous cell culturing module 14 is configured in the barotropic room 12 and comprises a culture vessel 141 and a feed pipe 142, wherein the culture vessel 141 is used for culturing the autologous cells into cultured autologous cells, and the feed pipe 142 is connected to the culture vessel 141 for receiving the cultured autologous cells. The supplying module 16 is configured in the barotropic room 12 and comprises a peristaltic feeder 161 and a discharge pipe 162, wherein the peristaltic feeder 161 is connected to the feed pipe 142 for receiving the cultured autologous cell and mixing the cultured autologous cells with a polymer material to be combined autologous cells, and the discharge pipe 162 is connected to the peristaltic feeder 161 for receiving the combined autologous cells. The multi-axis molding module 18 is configured in the barotropic room 12, and the multi-axis molding module 18 is connected to the discharge pipe 162 for receiving the combined autologous cells and customizing to print the combined autologous cells to the artificial implants. The implant saving module 19 is configured in the barotropic room 12 and is set under the multi-axis molding module 18 for carrying and saving the artificial implants. Wherein, the feed pipe 142 and the discharge pipe 162 are enclosed pipelines respectively.

Furthermore, the connection between the feed pipe 142 and the culture vessel 141, the connection between the peristaltic feeder 161 and the feed pipe 142, the connection between the discharge pipe 162 and the peristaltic feeder 161 and the connection between the multi-axis molding module 18 and the discharge pipe 162 are enclosed pipelines respectively.

Wherein, the gas inputted into the barotropic room 12 is a gas suitable for culturing the autologous cells.

The autologous cell culturing module 14 further comprises a motor 143, and the motor 143 is connected to the culture vessel 141 for driving the culture vessel 141 to produce a centrifugal rotation.

In the practice application, appropriate gas and centrifugal rotation can promote autologous cells to accelerate the growth and separate the cultured autologous cells.

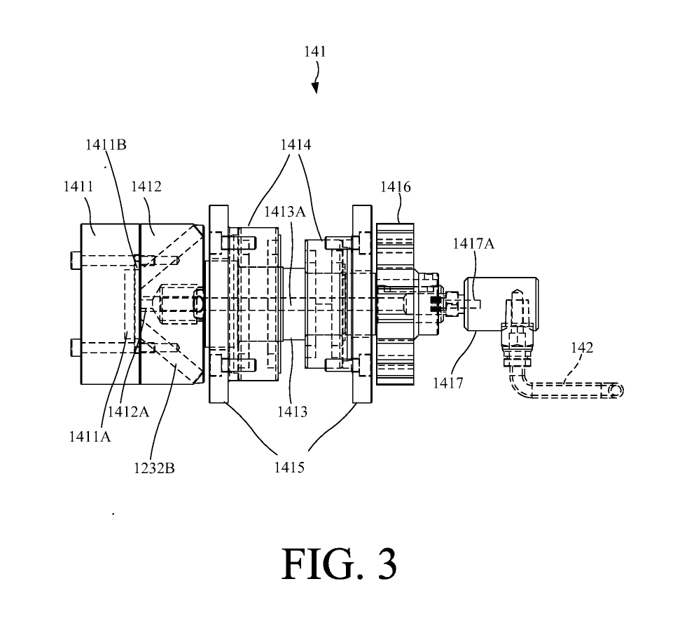

Please refer to FIG. 3. FIG. 3 shows a schematic diagram of the culture vessel 141 of the autologous cell multidimensional molding apparatus 1 in an embodiment of the present invention.

A culture vessel cover 1411 is combined with a culture vessel base 1412 to form a cell culturing space 1411A, and a gasket 1411B is between the two of them to form an adhered state. The bottom of the culture vessel base 1412 is combined with a transmission shaft 1413, and the two of them are connected to each other via a connected channel I (1412A) and a connected channel II (1413A), so that the cultured autologous cells can be outputted from the culture vessel 141. When the cultured autologous cells are outputted from the cell culturing space 1411A, a ventilation shaft 1412B is used for keeping the cell culturing space 1411A in pressure balance. A bearing group 1414 and a fixed cover group 1415 are placed in an appropriate portion of the transmission shaft 1413 for the rotation of the transmission shaft 1413, thereby enabling the autologous cells in the culture vessel 141 to achieve a gravity-free environment for the rapid growth of the autologous cells. The transmission shaft 1413 is combined with a transmission gear 1416 to obtain the power required for the rotation. The end of the transmission shaft 1413 is connected to a rotary joint 1417, and the two of them are connected to each other via the connected channel II (1413A) and a connected channel III (1417A), so that the cultured autologous cells in the culture vessel 141 can be delivered from the feed pipe 142 to the peristaltic feeder 161.

In the practice application, the suction power of the peristaltic feeder 161 can draw the cultured autologous cells from feed pipe 142 to the peristaltic feeder 161 and mix the cultured autologous cells with the appropriate polymeric material to be desired mixed autologous cells. Then, the compression power of the peristaltic feeder 161 delivers the mixed autologous cells to the multi-axis molding module 18 via the discharge pipe 162.

The multi-axis molding module 18 comprises a printing material injection head 181, and the printing material injection head 18 is connected to the discharge pipe 162 for inputting the combined autologous cells and printing the artificial implants. Wherein, the multi-axis molding module 18 is a six-axis molding module, the six-axis molding module comprises a pattern receiving element 182 and a six-axis moving arm 183, the pattern receiving element 182 is used for receiving a customized pattern, and the six-axis moving arm 183 is electrically connected to the pattern receiving element 182 for driving a customized print itinerary of the printing material injection head 181.

Furthermore, the pattern receiving element 182 can receive the customized pattern via Bluetooth, Ethernet, Wi-Fi, wireless mobile communication modules, or other accessible mobile storage devices.

The above-mentioned six-axis moving arm 183 moves in a three-dimensional space with an angular movement.

In the practice application, the mixed autologous cells are draped over the artificial tissue structure to allow the cells to grow on the structure to form artificial implants through the six-axis moving arm 183 moving in a three-dimensional space with an angular movement.

The implant saving module 19 has an enclosed storage environment suitable for the artificial implants and is a mobile device.

An environment of pollution-free is produced through the implant saving module 19 placed in the barotropic room 12, and the suitable sterile closed storage environment of the implant saving module 19 is used to effectively avoid the contamination of the artificial implants to ensure that the artificial implants have certain activity and efficacy. In the practice application, the duration from production completed to patients' actual use of the artificial implants (such as artificial dressings, artificial blood vessels and artificial breasts) is needed to wait for patients to arrive at the site or move the artificial implants to the appropriate site However, the implant saving module 19 can save the artificial implants to be used in the way of delivering the artificial implants to remote areas or in portable way, so that the artificial implants are more flexible in situations and time when used.

As compared to the prior art, the autologous cell multidimensional molding apparatus of the present invention uses the barotropic room to produce a production environment of pollution-free, uses the closed connection to isolate the whole production process from the autologous cell material to the artificial implant product from the outside world, and uses the six-axis molding module to make a customized autologous cell multidimensional print to get the high yield of the customized artificial implants. Also, the implant holding device can be used to make an artificial implant of flexible time and place.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. An autologous cell multidimensional molding apparatus, for customizing an artificial implant by using an autologous cell, comprising:
a barotropic room, for inputting a gas having a pressure greater than an ambient pressure;
an autologous cell culturing module, configured in the barotropic room, comprising a culture vessel and a feed pipe, wherein the culture vessel is used for culturing the autologous cell into a cultured autologous cell, and the feed pipe is connected to the culture vessel for receiving the cultured autologous cell;
a supplying module, configured in the barotropic room, comprising a peristaltic feeder and a discharge pipe, wherein the peristaltic feeder is connected to the feed pipe for receiving the cultured autologous cell and mixing the cultured autologous cell with a polymer material to be a combined autologous cell, and the discharge pipe is connected to the peristaltic feeder for receiving the combined autologous cell;
a multi-axis molding module, configured in the barotropic room, wherein the multi-axis molding module is connected to the discharge pipe for receiving the combined autologous cell and customizing to print the combined autologous cell to the artificial implant; and
an implant saving module, configured in the barotropic room, set under the multi-axis molding module for carrying and saving the artificial implant;
wherein the feed pipe and the discharge pipe are enclosed pipelines respectively.

2. The autologous cell multidimensional molding apparatus of claim 1, wherein the connection between the feed pipe and the culture vessel, the connection between the peristaltic feeder and the feed pipe, the connection between the discharge pipe and the peristaltic feeder, and the connection between the multi-axis molding module and the discharge pipe are enclosed pipelines respectively.

3. The autologous cell multidimensional molding apparatus of claim 1, wherein the gas inputted into the barotropic room is a gas suitable for culturing the autologous cell.

4. The autologous cell multidimensional molding apparatus of claim 1, wherein the autologous cell culturing module comprises a motor, and the motor is connected to the culture vessel for driving the culture vessel to produce a centrifugal rotation.

5. The autologous cell multidimensional molding apparatus of claim 1, wherein the multi-axis molding module comprises a printing material injection head, and the printing material injection head is connected to the discharge pipe for inputting the combined autologous cell and printing the artificial implant.

6. The autologous cell multidimensional molding apparatus of claim 5, wherein the multi-axis molding module is a six-axis molding module, and the six-axis molding module comprises a pattern receiving element and a six-axis moving arm, and the pattern receiving element is used for receiving a customized pattern, and the six-axis moving arm is electrically connected to the pattern receiving element for driving a customized printing itinerary of the printing material injection head.

7. The autologous cell multidimensional molding apparatus of claim 6, wherein the pattern receiving element can receive the customized pattern via Bluetooth, Ethernet, Wi-Fi, wireless mobile communication modules, or other accessible mobile storage devices.

8. The autologous cell multidimensional molding apparatus of claim 6, wherein the six-axis moving arm moves in a three-dimensional space with an angular movement.

9. The autologous cell multidimensional molding apparatus of claim 1, wherein the implant saving module has an enclosed storage environment suitable for the artificial implant.

10. The autologous cell multidimensional molding apparatus of claim 9, wherein the implant saving module is a mobile device.
